(19) United States
(12) Patent Application Publication
(10) Pub. No.: US 2023/0173161 A1
(43) Pub. Date: Jun. 8, 2023

(54) Conveying Device for Body Fluid

(71) Applicant: MEDELA HOLDING AG, Baar (CH)

(72) Inventor: Hilmar Ehlert, Hergiswil (CH)

(21) Appl. No.: 17/999,840

(22) PCT Filed: May 25, 2021
(86) PCT No.: PCT/EP2021/063816

§ 371 Date: Nov. 23, 2022

(30) Foreign Application Priority Data
May 27, 2020 (EP) 20176822.3

(51) Int. Cl. A61M 1/00

(52) U.S. Cl. CPC A61M 1/74; A61M 1/842; A61M 2205/103

(57) ABSTRACT

A conveying device for body fluid includes a catheter tube having a distal end to be inserted into the body and a proximal end. In order to provide a conveying device which can no longer become clogged with coagulating substances, a hollow body which is arranged in a longitudinally moveable and/or rotatable manner in the catheter tube, extends from the distal end at least up to the proximal end, and forms a transport channel over its length extending in the extension direction of the catheter tube.

The present invention relates to a body fluid conveying device comprising a catheter tube having a distal end to be inserted into the body and a proximal end.

Such a conveying device is known from WO 2017/152125 A2, for example. The solution known from this prior art was proposed in view of the problem that a conveying device in the form of a catheter tube which sucks body fluid from the chest region and in the course of a surgical intervention on the heart can become clogged. The solution previously known from WO 2017/152125 A2 proposes to connect the proximal end of the catheter tube inserted into the body to several tubes laid in parallel so that one tube can be used as a drainage tube for draining the body fluid, for example, while another tube can be flushed by introducing a flushing medium, for example via a third tube. This is intended to prevent clogging of the conveying system by coagulating blood drops or other body substances.

However, the previously known solution cannot prevent the catheter tube absorbed in the body from becoming clogged. The measures proposed in accordance with WO 2017/152125 A2 only apply to that part of the conveying device which lies outside the body.

The underlying problem of the present invention is to provide a conveying device of the type mentioned above, which counteracts in an improved manner the clogging of the catheter tube by coagulating substances from the body.

In order to solve this problem, the present invention provides a conveying device with the features of claim 1.

The conveying device according to the invention has a hollow body which is arranged longitudinally moveable and/or rotatable, in particular freely rotatable, in the catheter tube and extends from the distal end at least up to the proximal end of the catheter tube, if necessary also beyond it. Accordingly, the hollow body penetrates the entire catheter tube. The hollow body is preferably configured as an elongated bending body or shaft, i.e. it can be bent in the longitudinal direction without impairing the rotatability of the hollow body in the catheter tube. The bendability in this sense is preferably at least 90°.

According to the invention, the hollow body is hollow in the extending direction of the catheter tube. This cavity extends in the extending direction of the catheter tube and forms a transport channel therein. Thus, despite the presence of the hollow body, fluid can be transported through the catheter tube, for example by suction at the proximal end. The hollow body can form the aforementioned cavity in its interior. The outer dimension of the hollow body can correspond approximately to the inner diameter of the catheter tube. In this case, the cavity is usually formed along the central longitudinal axis of the hollow body.

The hollow body per se can have elastic and restoring properties. It is preferably formed by a plurality of windings or has discrete spacing elements which extend radially in relation to the longitudinal extension of the hollow body. When pressure is applied from outside, the hollow body ensures a certain amount of freedom for the catheter tube while maintaining the transport channel so that it cannot kink and be misplaced. This makes the practice of placing several catheter tubes leading to the outside, for example for the drainage of the pericardium, obsolete in order to ensure sufficient drainage even if one of the tubes is misplaced. The catheter tube can also be configured with thinner walls than before.

The hollow body can also improve the transport of corpuscular components, such as coagulate, through the catheter tube. Depending on the flow conditions within the catheter tube, such coagulate can be sheared and minimized by partially laying the catheter tube on it.

The hollow body is preferably configured with a helical scraping segment which extends radially from a rotation axis of the hollow body. When the hollow body is rotated and/or longitudinally moved within the catheter tube, coagulated blood drops adhering between the hollow body and the inner shell surface of the catheter tube break up mechanically so that a suction source connected to the catheter tube can better suck the fluid provided in the catheter tube out of it. Several scraping segments can also be arranged one behind the other.

Such scraping segments are usually provided distributed over the entire axial length of the hollow body so that the coagulating substances can basically be scraped over the entire length of the catheter tube.

The transmission of the movement of the hollow body, for example of a torque to the hollow body, can be done in different ways. For example, the hollow body can be driven contact-free and, for example, magnetically by providing a magnetic drive element on the outer circumferential surface of the catheter tube and connecting this magnetic drive element to the hollow body at the appropriate position, preferably in the region of the proximal end of the hollow body. The hollow body preferably protrudes beyond the free end of the catheter tube, where it is drivingly connected to a drive.

It is preferred to form the hollow body by means of a helix so that the rotation of the hollow body in the catheter tube also leads to the conveying of the contents of the catheter tube in the manner of an Archimedean screw. The helix can have an outer diameter which corresponds approximately to the inner diameter of the catheter tube so that the helix completely scrapes against the catheter tube when rotating.

The catheter tube preferably has an outer diameter of 16 to 36 French corresponding to 5.33 mm and 12 mm. The wall thickness of the catheter tube is usually between 0.5 and 2 mm. The inner diameter of the catheter tube is usually between 4 mm and 15 mm, most preferably the upper limit of the inner diameter of the catheter tube is 10 mm. A radial play between the outer diameter of the hollow body and the inner diameter of the catheter tube is usually between 0.5 mm and 1 mm.

As mentioned above, the hollow body preferably has radially extending scraping segments over its entire length. These scraping segments protrude radially from an axis of rotation and/or longitudinal movement of the hollow body, for example with an axial distance to each other so that a rotation and/or longitudinal movement of the hollow body results in coagulated material adhering to the inner surface of the catheter tube being scraped off or that adhesion is prevented or made impossible from the outset. The scraping segments can be provided with an axial distance to each other, since the shearing of adhesions on the catheter tube caused by the scraping segments continues over a certain axial or circumferential length. If the hollow body is formed by a helix, the said scraping segments are each formed continuously one after the other by the outer circumferential surface of the helix which, when the hollow body is rotated by 360°, sweep past a certain position on the inner circumferential surface of the catheter tube once each time and thus scrape off adhesions on the surface of the catheter tube.

With regard to an improved conveyance, in particular of corpuscular components in body fluid upon entry into the catheter tube, it is proposed, according to a preferred further development of the present invention, to provide the catheter tube in the region of its distal end on its shell surface with at least one opening, preferably with several openings distributed around the circumference. In this preferred configuration, the hollow body has at least one scraping edge interacting with the opening so that upon rotation of the hollow body, corpuscular or cellular components of the body fluid to be sucked off lying in the opening can be broken down. Such configurations are generally known from WO 2005/084562 A2 or U.S. Pat. No. 6,264,667 B1, EP 470 888 B1 or U.S. Pat. No. 5,569,178 B. In contrast to this prior art, the conveying device according to the invention preferably has scraping segments distributed over the entire length of the hollow body, which extend radially with respect to the rotation axis of the hollow body, but in any case a hollow body which regularly moves the scraping edge relative to the opening. While in the aforementioned prior art corresponding configurations are intended to break down tissues or cells before entering the catheter tube, the conveying device according to the invention can additionally prevent particles from adhering to the inner circumferential surface of the catheter tube or break up such adhering particles by means of the scraping segments which are preferably evenly distributed over the length of the hollow body. The conveying device according to the invention enables the transport of solids which cannot be moved under the mere influence of vacuum. In the present solution, these are mechanically conveyed out by means of the hollow body, which is preferably configured with at least one helical scraping segment.

The catheter tube is usually open at the end so that fluid can be sucked axially into the catheter tube and can also be discharged axially from the catheter tube. Nevertheless, according to a preferred further development of the present invention, the catheter tube has an axial stop for the hollow body at its distal end. This axial stop usually surrounds the inlet opening into the catheter tube, which is aligned with the axially continuous cavity of the hollow body.

The transmission of a rotary and/or translatory movement of the hollow body is preferably achieved by a drive element into which the hollow body can be inserted, possibly also through which the hollow body can be passed and with which the hollow body can be coupled. The coupling is preferably effected inevitably when the hollow body is inserted into the drive element, for example by elastic deformation of the drive element and/or the hollow body. The hollow body is preferably inserted axially into the drive element.

According to a preferred further development of the present invention, the conveying device has a coupling unit with a coupling housing. This coupling housing has a catheter tube connection and a suction line connection. The catheter connection is adapted to the fluid-tight connection of the catheter tube. The catheter connection can be a Luer connection, another bayonet connection or an open tube end without a special connection. The catheter connection can also be formed by a connecting pin onto which the catheter can be pushed with its proximal end. The suction line connection, which can be designed in a similar way, communicates with a suction line that leads to a collection container for the sucked body fluid. For the development discussed here, a flow channel is provided in the coupling housing. This flow channel communicates with the catheter tube connection and the suction line connection. The flow channel is usually arranged between these two connections.

A drive element is preferably mounted on the coupling housing for rotary drive of the hollow body. In this way, the coupling unit creates a fluid-tight connection between the catheter tube and the suction line on the one hand, and enables the transmission of a driving force or torque from the drive element to the hollow body on the other. Within the coupling unit, this hollow body protrudes beyond the catheter tube and can therefore be driven directly mechanically. The drive element can be connected to the hollow body either permanently or only drivingly. The drive element is preferably configured in the form of a sleeve drive element, which is drivingly coupled with a sleeve connected to the hollow body in a non-rotating manner and can accommodate it within itself and/or drive it from the outside.

The drive element can form a curved path or be connected to one which interacts with a stationary counter surface, for example a cam, and lies against it, for example under pretension so that a rotary movement of the drive element inevitably also leads to an axial movement of the same and thus to a cyclical translatory movement of the hollow body in the catheter tube.

The present invention is based on the idea that even though the catheter tube can be cut to the desired length in the course of surgical treatment of a human being, the hollow body does not have to be cut. Thus, the suction line connection has an inner diameter adapted to the passage of the hollow body. Usually, after the hollow body has been drivingly coupled with the drive element, the hollow body protrudes through both the coupling unit and the suction line connection as well as a partial length of the suction line. Accordingly, the inner diameters of the connections for the catheter and the suction power or the drive element or a drive sleeve, which accommodates and couples the hollow body, are configured accordingly for passing through the hollow body.

The flow channel is, according to a preferred further development, at least partially formed by a sleeve, which is mounted rotatably and longitudinally movable in the coupling housing. This sleeve can be detachably connected to the hollow body, thus it can be coupled in the sense of the present invention. The sleeve can form the drive element or be connected to it only drivingly. The coupling can be effected, for example, by a grub screw or the like which penetrates the sleeve and acts against the hollow body. Alternatively, the sleeve can also be elastically movable radially within limits so that the hollow body can be pushed through the sleeve against a certain resistance, but nevertheless an essentially fixed, in particular non-rotatable connection between the hollow body and the sleeve is achieved in such a way that when the sleeve is driven, it transmits the driving force or the torque to the hollow body so that it rotates inside the catheter tube and/or is moved longitudinally therein, in particular cyclically to and fro. According to this, the hollow body is preferably connected to the sleeve by positive and/or non-positive locking. The sleeve is usually sealed in the coupling housing so that the sucked-off fluid can be transferred from the catheter tube into the suction line without leakage.

When assembled, the sleeve is usually completely accommodated within the coupling housing, wherein at least one of the tube connections can be detachably attached to the coupling housing and cover the sleeve within the coupling housing. The fluid-tight connection may be achieved by a seal provided between the detachable tube connection and the coupling housing. The sleeve can also be detachably drivingly connected to the drive element. This drive element can, for example, be a mechanical drive element, for example of a gearbox, or a drive element magnetically coupled to the sleeve. The drive element can act on a drive sleeve, which accommodates and surrounds the aforementioned sleeve. This drive sleeve can be provided with magnets or a toothing.

According to a preferred further development, a flushing line is provided which communicates with the catheter tube and/or the suction line or the flow channel. This flushing line can, for example, be connected directly to the catheter tube near its distal end to improve transport through the catheter tube by cyclic flushing. However, the flushing line can also be connected outside the body first. The catheter tube and/or the coupling unit or suction line can also have a flushing connection for the flushing line there. The flushing connection can be attached to the coupling housing, preferably on the catheter side of the drive unit, in order to be able to flush the drive unit as well. Usually, the flushing fluid introduced via the flushing line or the flushing connection is discharged via the suction line, thus, the conveying device is at least partially flushed.

The conveying device according to the invention preferably has a drive that can be coupled drivingly to the drive element. This coupling can be achieved, for example, by a flexible shaft or another torque transmission. In view of hygienic requirements, the drive is regularly provided as a separate unit to the coupling unit and is only drivingly coupled to the coupling unit. Thus the coupling unit can also be produced as a consumable part and disposed of after use. A drive control is assigned to the drive which is configured such that the hollow body can be driven continuously and/or reversing and/or cyclically by the drive. The hollow body can thus be driven in various ways, in particular to scrape off coagulate adhering to the circumferential surface of the catheter tube or to convey the clot (solid body) through the catheter purely mechanically.

The drive of the hollow body can preferably be effected depending on the degree of clogging of the catheter tube. For this purpose, the drive control usually has a power control, for example a torque detection. The torque detected by the latter, for example, is included in the drive control. If, for example, a relatively high power consumption is required on the side of the drive for the desired relative movement of the hollow body within the catheter tube due to considerable coagulation, the hollow body is driven for a longer period of time compared to an uncritical torque or other power recognition of the drive, which indicates that the catheter tube is at most slightly clogged.

According to a preferred further development of the present invention, the drive control for the drive is realized in a structural unit with a suction control of a suction pump. This suction pump is assigned in a manner known per se to a collection container which can be connected to the suction line. Such collection containers with associated suction pump and suction control are generally known for sucking off body fluids, for example from WO 2018/054833 A2. Due to the uniform configuration of drive control and suction control, the technical complexity for the production of the conveying device can be reduced. Moreover, the suction on the one hand and the operation of the hollow body on the other hand can be coordinated. A pressure sensor measuring the suction pressure can also be connected to the drive control in order, for example, to interpret an increased suction pressure as a measure of increasing displacement of the catheter tube and to control the drive to rotate the hollow body accordingly.

The conveying device according to the invention allows the effective conveying of body fluid out of the body without the risk of an increasing reduction in the cross-section of the catheter tube due to coagulating substances. These are not only drawn off, but also removed from the inner circumferential surface of the catheter tube by scraping from its inner circumferential surface over the entire axial length of the catheter tube, and are discharged from the catheter tube by sucking on the catheter tube and/or operating a conveying shaft element as a hollow body, such as a helix. The hollow body also causes the catheter tube to be elastically released so that it can be reliably transported through the catheter tube due to the cavity in the hollow body.

If, for example, a patient with blood in the pericardium is treated with the conveying device according to the invention, the catheter tube is first led into the pericardium. Then the catheter tube is cut to the desired length. Subsequently, the hollow body is inserted into the catheter tube. The catheter tube and the hollow body are usually made available to the attending physician as a sterilely packed supply unit. After cutting the catheter tube, the hollow body has a greater length than the catheter tube. The hollow body is inserted into the catheter tube up to the stop provided at the distal end.

The hollow body is then connected to the drive element, for example the sleeve, in a non-rotating manner, wherein the catheter tube connection has been pushed over the proximal end of the catheter tube beforehand.

If necessary, a radially extending spring, for example in the form of a grub screw or a clamping device, can be used to non-rotatably connect the hollow body to the sleeve.

The sleeve is then inserted into the coupling housing. In another configuration, the sleeve is an integral component of the coupling housing, which has a positive effect on the tightness of the system. In this case, the sleeve is usually permanently connected to the coupling housing. The catheter tube connection and the suction line connection are screwed to the coupling housing. The coupling housing usually contains a drive sleeve which thereby is connected to the sleeve in a non-rotating manner, for example via a tongue-and-groove connection, and is drivingly coupled to a drive. The sleeve can be an integral component of the coupling housing.

The hollow body then usually protrudes beyond the suction line connection and in any case is located in a partial length of the suction line. This suction line leads to a collection container of a suction device which also accommodates the suction pump, a control to the suction pump and the drive control to the drive. Fluid can be sucked out of the pericardium by operating the suction pump. A threatening clogging is prevented by operating the drive and rotating the hollow body in the catheter tube.

Further details and advantages of the present invention can be derived from the following description of an embodiment in conjunction with the drawing. Therein:

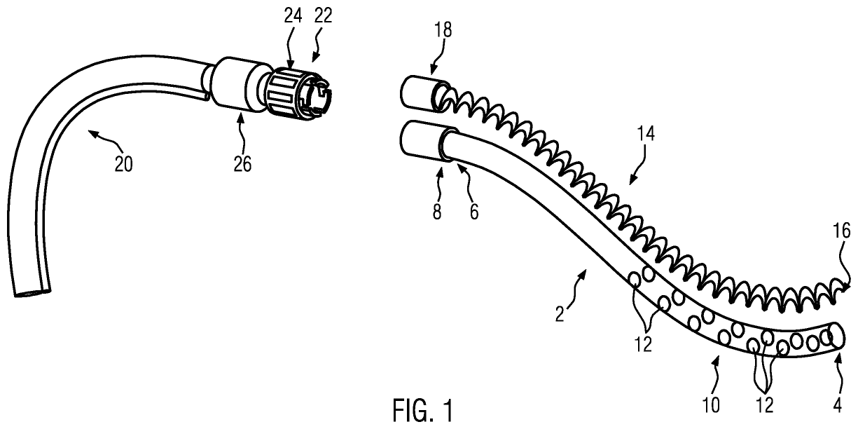

FIG. 1 shows a side view of essential components of the embodiment of a conveying device;

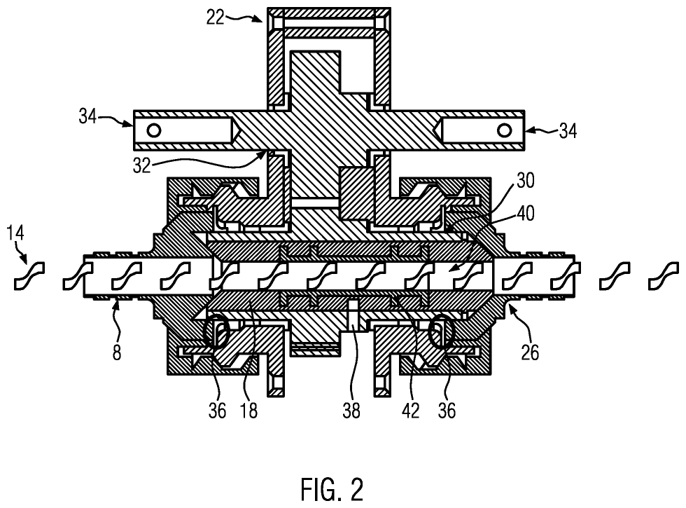

FIG. 2 shows a longitudinal sectional view of an embodiment of a coupling unit of the present invention; and

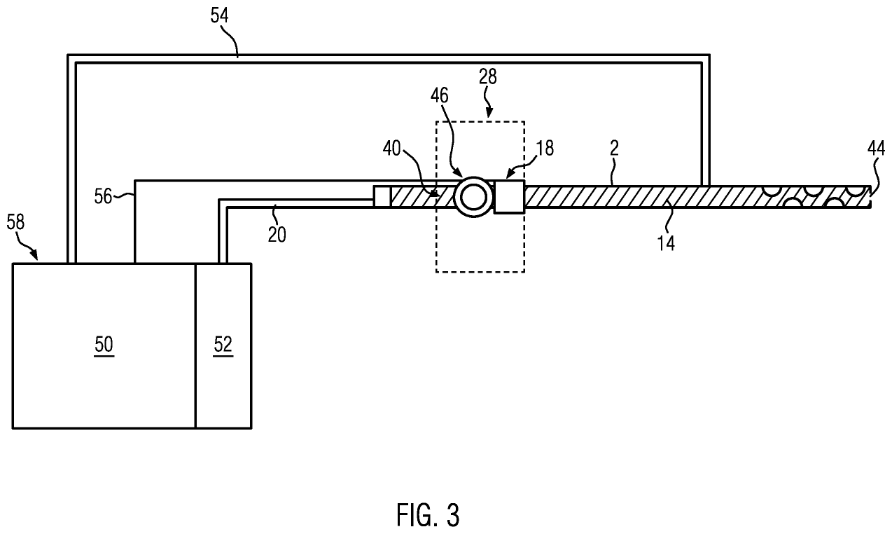

FIG. 3 shows a schematic view of functional components of the embodiment of the conveying device.

FIG. 1 shows a catheter tube 2 with a distal end 4 and a proximal end 6, wherein the proximal end 6 is provided with a catheter tube connection 8. The distal end region of the catheter tube 2, identified by reference sign 10, has on its shell surface a plurality of circumferentially and axially spaced openings 12. A hollow body is identified by reference sign 14, which in the present case is formed as a helix, wherein the individual windings of the helix leave free between them an inner, axially extending cavity which forms a transport channel 16. The distal end of the hollow body 14 extends to the distal end 4 of the catheter tube 2.

The proximal end of the hollow body 14 may be accommodated in a drive sleeve 18 and connected thereto in a non-rotating manner, or may project beyond this drive sleeve, which is an example of a drive element within the meaning of the present invention.

Between the catheter tube 2 and a tubular suction line 20, a coupling housing 22 with magnetic fields alternating in circumferential direction is provided at its end which form a magnetic drive 24. The coupling housing 22 is hollow inside to accommodate the drive sleeve 18. The coupling housing 22 is non-rotatably connected to the suction line 20. The drive sleeve 18 is provided with magnets assigned to the outer circumference of alternating fields. Reference sign 26 identifies a suction line connection with a cylindrical sleeve for accommodating the magnetic drive 24.

In the assembled state, the drive sleeve 18 is surrounded by the coupling housing 22 and the magnetic drive 24 attached to it which, due to the circumferentially alternating magnetic fields, rotationally drives the drive sleeve 18 which carries the hollow body 14 along.

FIG. 2 shows an alternative to driving the hollow body 14, wherein the illustration of the catheter tube 2 and the suction line 20 was omitted. The same components are identified with the same reference signs as in the previous embodiment. However, the embodiment also illustrates in detail tube connections for the catheter tube 2 and the suction line 20 compared to the example in Fig.

In the second embodiment according to FIG. 2, the coupling housing 22 is formed by a plastic body to which the catheter connection 8 and suction line connection 26 are detachably connected. In this embodiment, the catheter tube 2 and the suction line 20 can be simply plugged onto the catheter connection 8 or the suction line connection 26 with their open ends without additional connecting elements. The coupling housing 22 rotatably supports the drive sleeve 18 and a sleeve 30, which is non-rotatably connected to the drive sleeve 18, as well as a drive element in the form of a sleeve drive element 32. The sleeve drive element 32 has a toothing which meshes with a toothing on the sleeve 30. The sleeve drive element 32 has a drive shaft 34 which protrudes beyond the coupling housing 22 for drivingly coupling with a drive not shown, for example in the form of an electric motor.

The catheter connection 8 and the suction line connection 26 are screwed to the coupling housing 22 with the interposition of seals 36. Reference sign 38 indicates a grub screw which penetrates the drive sleeve 18 and abuts against the sleeve 30 in order to connect it non-rotatably to the hollow body 14.

FIG. 2 shows a flow channel 40, which is provided between the catheter connection 8 and the suction line connection 26 and is in any case partially formed by the drive sleeve 18. In the embodiment shown, the flow channel 40 is defined within the drive sleeve 18 over a certain partial length by a pretension sleeve 42, which is connected to the drive sleeve 18 in a non-rotating manner. Between the inner circumferential surface of the drive sleeve 18 and the outer circumferential surface of the pretension sleeve 42, a pretension means, for example in the form of a spring element or a compressible gas, may be provided which radially pretensions the pretension sleeve 42 such that the inner diameter formed by the pretension sleeve 42 is smaller than the outer diameter of the hollow body 14. When the hollow body 14 is passed through the pretension sleeve 42, a radial expansion of the hollow body 14 occurs, wherein a non-rotating connection between the drive sleeve 18 and the hollow body 14 inevitably results. The alternative described above represents a simple possibility of connecting the hollow body 14 to the drive sleeve 18 in a non-rotating manner by passing the hollow body 14 through the drive sleeve 18.

The non-rotating connection between the drive sleeve 18 and the sleeve 30 can also be achieved by grooves and tongues extending in the longitudinal direction of the flow channel 40 and arranged between them when the drive sleeve 18 is pushed axially into the sleeve 30. A grub screw 38 can therefore also be dispensed with.

The axial determination of the drive sleeve 18 is done by the two connections 8, 26 which accommodate the drive sleeve 18 in a fluid-tight manner in the coupling housing 22.

In the embodiment shown, the drive shaft 34 projects beyond the coupling housing 22 on opposite sides so that a connection with different couplings is possible.

FIG. 3 schematically shows further components of an embodiment. Identical components are identified with the same reference signs as in the previous examples.

FIG. 3 illustrates the status after the treatment of a patient with the catheter tube 2, in which the hollow body 14 is provided. The hollow body 14 extends distally to an axial stop identified by reference sign 44 which radially narrows the inlet opening at the distal end of the catheter tube 2, while still allowing entry into the catheter tube 2. The hollow body 14 apparently extends beyond the schematically indicated coupling unit 28 which in addition to the drive sleeve 18 accommodates a pressure sensor 46, which records the internal pressure in the catheter tube 2 or the flow channel 40 or a suction line 20. In any case, the signal of the pressure sensor 46 is indicative of the suction pressure within the catheter tube 2. This pressure sensor 46 can communicate data-wise with a control device 50 which controls both the drive of the coupling unit 28 and a suction pump, which is usually provided in a structural unit with the control device 50 and generates a negative pressure in the suction line 20, through which the liquid sucked off via the catheter tube 2 reaches a collection container 52.

The control device 50 controls the function of the suction pump as well as the control of a drive which drives the hollow body 14.

A flushing line is identified with reference sign 54 with which the suction line 20 can be flushed depending on the signals of the control device 50. In an alternative configuration, the flushing line 54 can also be positioned so that the fluid-carrying region of the coupling unit 28 or the catheter tube 2 can be at least partially flushed.

Reference sign 56 indicates a flexible drive shaft which connects a drive not shown to the drive shaft 34 inside a medical suction device 58 which accommodates the control device 50 and usually holds the collection container 52 in a detachable manner.

### REFERENCE SIGN LIST

2 catheter tube
4 distal end
6 proximal end
8 catheter tube connection
10 distal end region
12 openings
14 hollow body
16 transport channel
18 drive element-drive sleeve
20 suction line
22 coupling housing
24 magnetic drives
26 suction line connection
28 coupling unit
30 sleeve
32 drive element-sleeve drive element
34 drive shaft
36 seals
38 grub screw
40 flow channel
42 pretension sleeve
44 stop
46 pressure sensor
50 control device
52 collection container
54 flushing line
56 drive shaft
58 suction device

1. A conveying device for body fluid comprising a catheter tube having a distal end to be inserted into the body and a proximal end, wherein a hollow body which is arranged longitudinally moveable rotatable in the catheter tube, extends from the distal end at least up to the proximal end, and forms a transport channel over its length extending in the extension direction of the catheter tube.

2. The conveying device according to claim 1, the hollow body is provided over its length with at least one radially extending scraping segment.

3. The conveying device according to claim 1, the hollow body being formed by a helix.

4. The conveying device according to claim 1, characterized in that the catheter tube at least one opening in the region of its distal end on its shell surface and the hollow body forming a scraping edge which interacts with the opening.

5. The conveying device according to claim 1, the catheter tube having an axial stop for the hollow body at its distal end.

6. The conveying device according to claim 1, the hollow body being insertable into a drive element and coupled to the drive element.

7. The conveying device according to claim 1, further comprising a coupling unit with a coupling housing having a catheter tube connection and a suction line connection which communicate with a flow channel surrounded by the coupling housing.

8. The conveying device according to claim 7, further comprising a drive element rotatably supported on the coupling housing for driving the hollow body.

9. The conveying device according to claim 7, the suction line connection, the catheter tube connection and/or the drive element having an internal diameter which is configured in a manner adapted for the passage of the hollow body.

10. The conveying device according to one of claims 7, the flow channel being at least partially formed by a sleeve which is rotatably supported in the coupling housing and can be detachably connected to the hollow body in a non-rotating manner.

11. The conveying device according to claim 10, the sleeve being detachably connectable to the coupling housing and detachably drivingly connectable to a sleeve drive element.

12. The conveying device according to claim 11, the sleeve drive element having a drive shaft which is exposed at the coupling housing.

13. The conveying device according to claim 1, further comprising a flushing line communicating with at least one of the catheter tube, the suction line, or with the flow channel.

14. The conveying device according to claim 6, further comprising a drive which can be drivingly coupled to the drive element, and a drive control associated with the drive, which is configured such that the hollow body can be rotated via the drive.

15. The conveying device according to claim 14, the drive control having a torque detection and in that the detected torque enters the control of the drive.

16. The conveying device according to claim 14, the drive control being in a structural unit with a suction control of a suction pump which is assigned to a collection container which can be connected to the suction line.

17. The conveying device according to claim 14, the drive control being connected data-wise to a pressure sensor measuring a suction pressure in the suction line.

18. A method for operating a fluid conveying device for body fluid comprising a catheter tube having a distal end and a proximal end and a hollow body which hollow body is arranged longitudinally moveable and/or rotatable in the catheter tube, which hollow body extends from the distal end at least up to the proximal end, and forms a transport channel over its length extending in the extension direction of the catheter tube in which method the hollow body is moved longitudinally and/or rotated within the catheter tube while the catheter tube is held in place and while a negative pressure is applied at the proximal end of the catheter tube to convey by means of a suction pressure liquid through the transport channel and into a collection container.

19. The conveying device according to claim 1, further comprising a flushing line communicating with the catheter tube and with the suction line and with the flow channel.
